(19) United States
(12) Patent Application Publication
(10) Pub. No.: US 2016/0367313 A1
(43) Pub. Date: Dec. 22, 2016

(54) IRRIGATED ABLATION CATHETER

(71) Applicant: OSYPKA AG, Rheinfelden (DE)

(72) Inventors: Claus-Christian ROHLIG, Rheinfelde (DE); Thorsten GOTTSCHE, Rheinfelde (DE)

(73) Assignee: Osypka AG, Rheinfelden (DE)

(21) Appl. No.: 15/219,863

(22) PCT Filed: Feb. 4, 2015
(86) PCT No.: PCT/EP2015/000215

§ 371 Date: Jul. 26, 2016

(30) Foreign Application Priority Data
Feb. 27, 2014 (EP) EP14000698.2

(51) Int. Cl. A61B 18/14

(52) U.S. Cl. CPC A61B 18/1492; A61B 2018/00964

(57) ABSTRACT

The present invention relates to catheters in general and more particularly to improvements in so-called “irrigated ablation catheter” that may be used in mapping and ablation procedures of tissue, preferably heart tissue in the treatment of cardiac arrhythmia. The present invention is directed irrigated ablation catheter comprising an elongated catheter shaft (4), a deflectable section (3) distal to the catheter shaft, an ablation catheter tip (1) distal to the deflectable section. The ablation catheter tip comprises an irrigation fluid flow distributor (10) and a cap(20) having a cylindrical side part and a tip, the flow distributor (10) has a distal end (16) and a proximal end (15) an inner channel (11) and an outer channel (12), whereby the distal end of the inner channel is coupled to the distal end of the outer channel, characterized in that the outer channel (12) has a helical shape in form of a double helix and is defined by the cylindrical part of the cap (20) and the irrigation fluid flow distributor (10), whereby the cylindrical part of the cap (20) is tightly slid over the irrigation fluid flow distributor (10) and seals the outer channel of the flow distributor thus forming two helical cavities; whereby irrigation fluid exit ports (21) are arranged on the cylindrical part of cap (20), said exit ports being arranged such that they follow the spiral course of the outer channel and whereby the cap has further irrigation fluid exit ports (23) which are arranged on the tip of the cap.

The present invention relates to catheters in general and more particularly to improvements in so-called “irrigated ablation catheter” that may be used in a mapping and ablation process of tissue, preferably heart tissue in the treatment of cardiac arrhythmia.

Irrigated catheters are commonly used in an ablation process. Irrigation provides many benefits including cooling of the electrode and tissue which prevents overheating of tissue that can otherwise cause the formation of coagulum.

An ablation catheter typically carries one or more electrodes, which are used for ablation. A common method used for ablation is the radiofrequency (RF) which is accomplished by transmission of radiofrequency energy to a desired target area through an electrode assembly to ablate tissue at the target site. The RF energy delivered through the electrode causes the tissue in contact with the electrode to heat. A problem which may occur is tissue overheating which causes unwanted tissue damage, uncontrolled lesion size and depth and the formation of a coagulum around the RF electrode which in turn reduces the efficiency of the ablation.

Known irrigated ablation catheters are, for example, open ablation catheters which deliver the cooling fluid through holes arranged on the surface of the ablation catheter tip to prevent overheating of the tissue and to achieve deeper lesion and reduced coagulum formation.

U.S. Pat. No. 6,611,699 describes various catheter designs with irrigated tip electrodes.

When placing the so called “open ablation catheters” to obliterate the area causing the arrhythmia it may happen that the irrigation fluid exit ports arranged on the surface of the ablation catheter tip are covered or blocked by tissue resulting in a hindered flow of irrigation fluid. The distribution of the cooling fluid in the inner cavity of the ablation electrode influences the temperature distribution on the surface of the electrode.

U.S. 20050055020 describes a helical ablation electrode. The electrode is a single helix, the cooling fluid passes the helix and exits at the fluid exit ports. In one embodiment shown in FIG. 5b, 6b the electrode comprises a tip around which the helical electrode winds. A fluid delivery lumen runs through the whole helical electrode delivering cooling fluid to cool the electrode. The cooling fluid follows the helical shape of the electrode. There are a number of fluid exit ports distributed over the electrode, thus following the helical shape.

U.S. 20070066878 describes a helical ablation electrode being covered by a porous cap said electrode further comprising an irrigation fluid flow distributor having a plurality of irrigation fluid exit ports. The irrigation fluid flow distributor is formed as straight tube. The exit ports are arranged along the tube preferably such that an irrigation port lies between each wind of the coil electrode.

U.S. Pat. No. 7,104,989 describes a helical virtual ablation electrode being covered by a non-conductive cap to form a fluid chamber between the cap and the electrode, said electrode further comprising a fluid flow distributor being formed as fluid trunk with one or more fluid distribution branches extending from the fluid trunk. The cap has a plurality of pores. When fluid is delivered through the fluid distribution branches the fluid fills the fluid chamber and flows out from the chamber through the plurality of pores.

U.S. 2011/0160726 describes a tip electrode in form of a cap and a fluid flow distributor being formed as a straight tube ending in an inner lumen following the hemispherical shape of the electrode tip thus forming a fluid chamber. Fluid exit ports are arranged on the electrode tip and connected to the fluid chamber.

There is still a need to provide an irrigated ablation catheter having an improved cooling system for reducing the overall electrode temperature even if irrigation exit ports on the ablation catheter tip are blocked. It should in particular be ensured that the heat removal from the catheter tip is permanent and continuous during the ablation process so that a homogeneous and effective cooling of the tip surface can be reached and maintained.

Especially desirable is an improved irrigation fluid flow distribution resulting in a homogeneous cooling of the ablation catheter tip thus avoiding the formation of hot spots at the surface of the ablation catheter tip, said tip being in direct contact with the tissue. The irrigation and cooling leads to a deeper and better penetration of the radiofrequency energy, the thermal effects of ablation are thus deeper and the result of ablation procedure is improved.

The present invention is directed to an irrigated ablation catheter comprising an elongated catheter shaft (4), a deflectable section (3) distal to the catheter shaft, an ablation catheter tip (1) distal to the deflectable section, the ablation catheter tip (1) comprises an irrigation fluid flow distributor (10) and a cap (20) having a cylindrical side part and a tip, the flow distributor (10) has a distal end (16) an inner channel (11) and an outer channel (12), whereby the distal end of the inner channel is coupled to the distal end of the outer channel, characterized in that the outer channel (12) has a helical shape in form of a double helix and is defined by the cylindrical part of the cap (20) and the irrigation fluid flow distributor (10), whereby the cylindrical part of the cap (20) is tightly slid over the irrigation fluid flow distributor (10) and seals the outer channel of the flow distributor thus forming two helical cavities; whereby irrigation fluid exit ports (21) are arranged on the cylindrical part of cap (20), said exit ports being arranged such that they follow the spiral course of the outer channel and whereby the cap has further irrigation fluid exit ports (23) which are arranged on the tip of the cap.

In use irrigation (cooling) fluid is pumped through the inner channel (11) leaving the inner channel at the distal end (16) of the irrigation fluid flow distributor (10). The cooling fluid then follows the spiral course of the helical outer channel (12). As the outer channel is in direct contact with a plurality of irrigation fluid exit ports (21, 23) whereby the exit ports (21) are arranged such that they follow the spiral course of the outer channel, the irrigation (cooling) fluid can exit the ablation catheter tip through the exit ports (21, 23) to cool the tissue adjacent to the ablation catheter tip.

The inventive concept is the combination of two structural parts, namely the helical construction of the irrigation outer channel combined with the corresponding position of the irrigation exit ports. Due to this concept, the homogeneity and effectiveness of the tissue cooling is guaranteed. Furthermore this concept allows setting the desired cooling properties. A permanent and continuous heat removal avoiding the formation of hot spots on the surface of the ablation electrode is ensured.

The ablation electrode according to the present invention comprises a flow distributor and a cap. The cap has a cylindrical part and a tip. The irrigation flow distributor has an inner channel and an outer channel being a double helix whereby the double helical shape is defined by the cylindrical part of the cap which is tightly slid over the irrigation flow distributor. Forming the outer channel in form of a double helix is done by milling. The cap over the outer channel is essential to define a tube like channel for the cooling fluid to be passed.

The helical outer channel consists of two helices which are wound around each other thus providing a double helix. Concerning the double helix the water flow of each helix reaches the same cooling point. This may be advantageous.

In one embodiment the cross section of the helical outer channel decreases from its distal to its proximal end to guarantee an optimal and constant, flow rate of the irrigation (cooling) fluid as the cooling fluid moves downwards.

Typically the flow rate of the cooling fluid is in the range between 2 m/minute and 20 ml/min.

Instead of decreasing the cross section, it would also be possible to vary the pitch of the helix.

The cap and the irrigation fluid flow distributor are in one embodiment made of the same conductive material, for example Au, Pt/Ir, Pt, stainless steel, conductive plastic and the like.

The cap and the irrigation fluid flow distributor are in one embodiment made of different conductive material, for example the cap is made of Au or Pt/Ir and the flow distributor is made of stainless steel.

In a further embodiment only the cap is made of a conductive material such as e.g. Au or Pt/Ir and the fluid flow distributor is made of a nonconductive material such as biologically compatible plastic.

Important is the position and distribution of the irrigation fluid exit ports arranged on the cap. Concerning the cap two areas are differentiated, the cylindrical side part of the cap and the tip of the cap. In both areas there are irrigation fluid exit ports.

The term “plurality of irrigation fluid exit ports” refers to more than one exit, preferably more than 10 exits, and more preferably more than 20 exits, for example 20-40 exits.

The irrigation fluid warms up to during the passage through the catheter device to finally reach body temperature.

The irrigation flow passage is such that cold fluid, which is supplied via the inner channel, passes at first the distal extremity of the flow distributor. Subsequently the cold fluid enters the cavity of the upper part of the cap and exits through the upper exit ports which are in direct and intense contact with the tissue to be ablated thus providing the tissue area of the most intense contact with the fluid which has only minimal warmed up. The cooling fluid then follows the spiral course of the outer channel thus evenly supplying the outer channel with cooling fluid. On each turn of the spiral course there are exit ports for the exit of the cooling fluid.

In case of exit ports being blocked by tissue, the heat removal is ensured by the cooling fluid following the spiral course of the outer channel, thus forcing the steady cooling of the inner surface of the electrode cap.

These and other features and advantages of the present invention will be better understood by reference to the following description when considered in conjunction with the accompanying drawings wherein:

FIG. 1 shows an ablation catheter device having the inventive irrigated ablation electrode.

FIG. 2A is a side cross sectional view of the catheter shaft having the inventive irrigated ablation electrode.

FIG. 2B is a cross sectional view of the catheter shaft taken along the line A-A of FIG. 2A.

FIG. 3A and FIG. 3B is a side view of the helical irrigation fluid flow distributor.

FIG. 4A and FIG. 4B is a side view of the cap.

FIG. 5A and FIG. 5B show the ablation catheter tip mounted on an adapter.

The numbering of the figures is as follows

1 Ablation catheter tip

2 Sensing electrode

3 Deflectable section

4 Catheter shaft

5 Fluid source

6 Energy source

7 Catheter handle

8 Electric conductive wire

9 Taut wire

10 Irrigation fluid flow distributor

11 Inner channel

12 Outer channel

14 Adapter

15 Proximal end of the irrigation fluid flow distributor

16 Distal end of the irrigation fluid flow distributor

20 cap

21 Exit ports in the side area

22 Temperature sensor

23 Exit ports in the tip area

### DESCRIPTION OF THE FIGURES

FIG. 1 shows an ablation catheter device having the inventive irrigated ablation electrode. The catheter comprises an elongated catheter shaft (4) a deflectable section (3) distal to the catheter shaft (4), an irrigated ablation electrode (1) which is positioned distal to the deflectable section (3). In use the ablation electrode is in direct and intense contact with the tissue to be ablated. Optionally there are one or more sensing electrodes (2) positioned on the deflectable section (3). Catheter handle (7) supports the proximal end of the catheter shaft (4). The energy source (6) is connected to the catheter handle (7) to supply energy to the ablation electrode (1). A fluid source (5) is attached to the catheter handle (7) to apply cooling fluid. The ablation catheter device is a device commonly used in ablation therapy. New and inventive is the design of the ablation electrode (1).

FIG. 2A shows the catheter shaft (4) and the deflectable section (3) having the inventive irrigated ablation electrode (1) and in addition sensing electrodes (2). The side cross-sectional view of the catheter shaft shows the electric conduction wires (8), an inner channel (10) for delivering the cooling fluid (irrigation fluid), a taut wire (9) for converting the ablation catheter from a retracted operation mode into an expanded operation mode.

FIG. 2B is a cross sectional view of the catheter shaft taken along the line A-A of FIG. 2A showing the tube (10) with the inner channel (11) for delivering the cooling fluid, the taut wires (9), the electrical conduction wires (8).

FIG. 3A and FIG. 3B is a side view of the helical irrigation fluid flow distributor (10)

FIG. 3A is an isometric view of the irrigation fluid flow distributor (10). The inner channel (11) delivering the irrigation fluid leads distally to the helical outer channel (12) which is in shape of a double helix. In use the irrigation fluid runs through the inner channel, leaves the inner channel at its distal end (16) and follows the spiral course of the outer channel starting at its distal end and running to its proximal end (15).

The flow distributor (10) is mounted via an adapter (14) to the deflectable section (3) of the catheter shaft (4).

FIG. 3B is a side view of the irrigation flow distributor (10). FIG. 3B shows in addition to FIG. 3A that the cross section of the helical outer channel decreases from its distal to its proximal end.

FIG. 4A and FIG. 4B is a side view of the cap (20). A plurality of irrigation fluid exit ports (21) are arranged on the cap wherein the exit ports are arranged such that they follow the spiral course of the outer channel of the irrigation flow distributor which is positioned inside the cap (not shown in this figure.) In the isometric view 4A a temperature sensor (22) is positioned within the distal portion of the tip of the cap.

FIG. 5A and FIG. 5B show the ablation catheter tip (1) mounted on adapter (14).

FIG. 5A is a side view showing the ablation catheter tip (1) mounted on adapter (14). The ablation catheter tip is as shown in FIG. 4b

FIG. 5B is a cross sectional view of the inventive ablation catheter tip (1) formed in two pieces, a cap (20) and an irrigation fluid flow distributor (10) having a distal end (16) and a proximal end (15). The flow distributor has an inner channel (11) and an outer channel (12). The inner channel (11) leads distally to the outer channel (12). The outer channel is in shape of a double helix having two turns. The cylindrical part of cap (20) is tightly slid over the irrigation flow distributor. The irrigation fluid exit ports are arranged on the cap. There are exit ports (23) arranged on the tip of the cap and exit ports (21) arranged such that they follow the spiral course of the outer channel. The cross section of the helical outer channel decreases towards the proximal end.

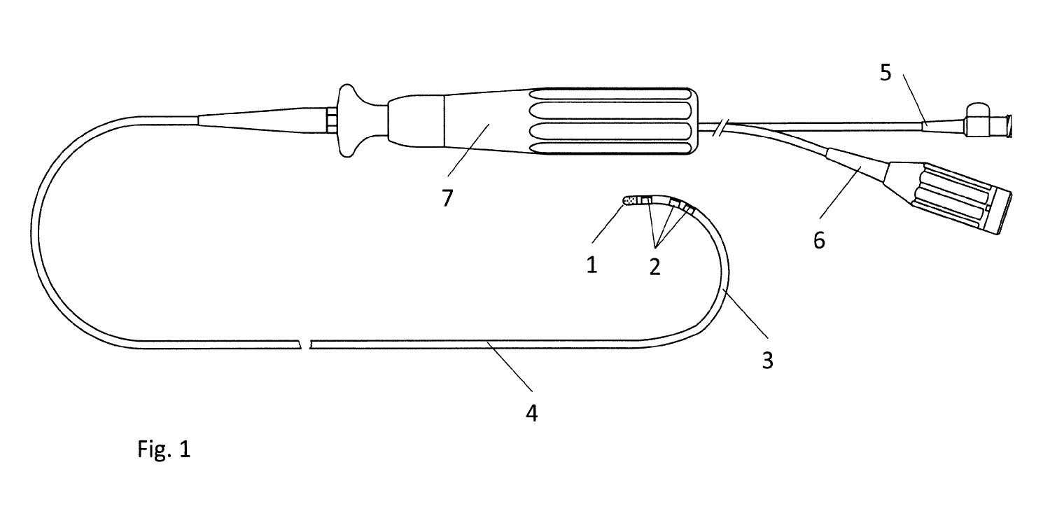

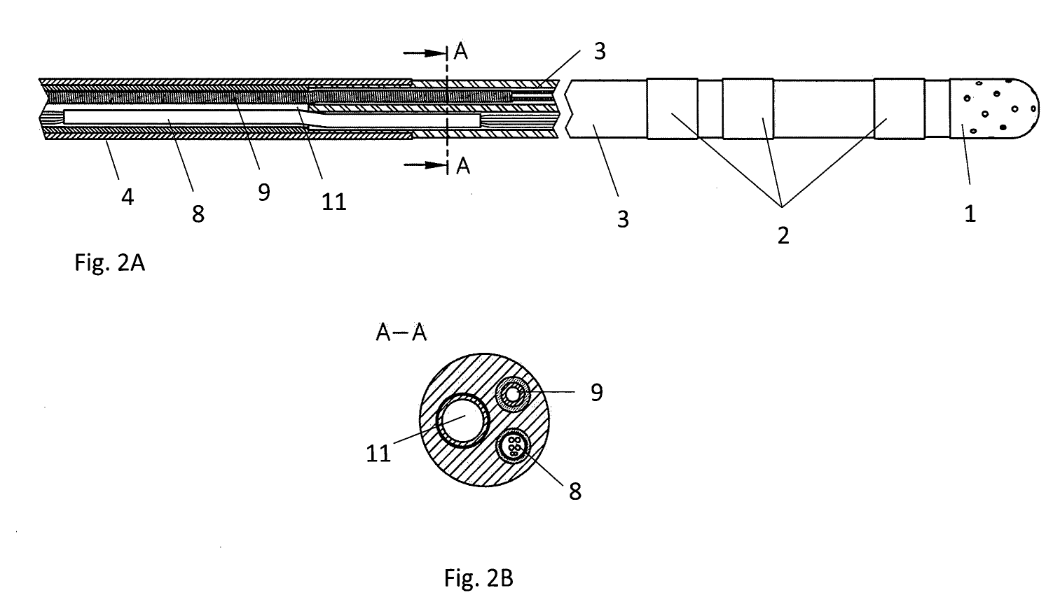

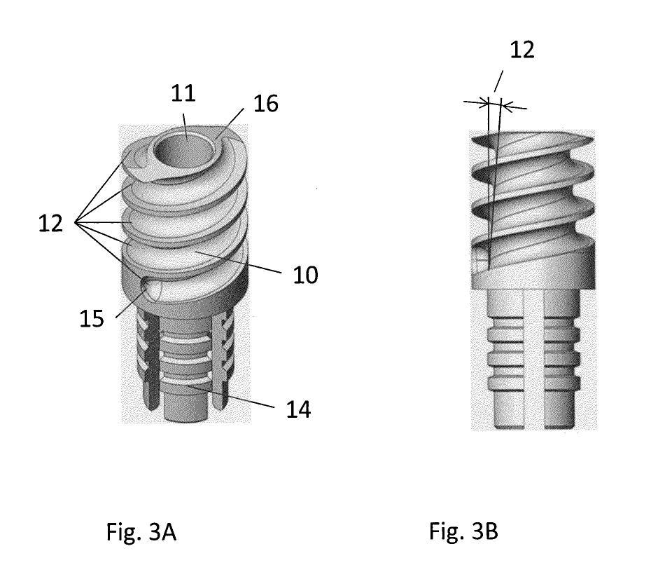

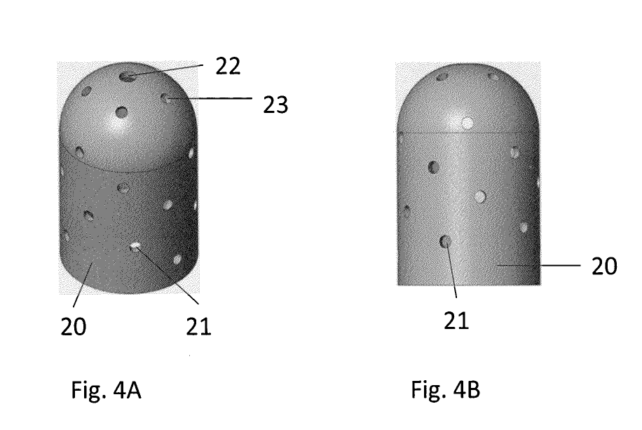

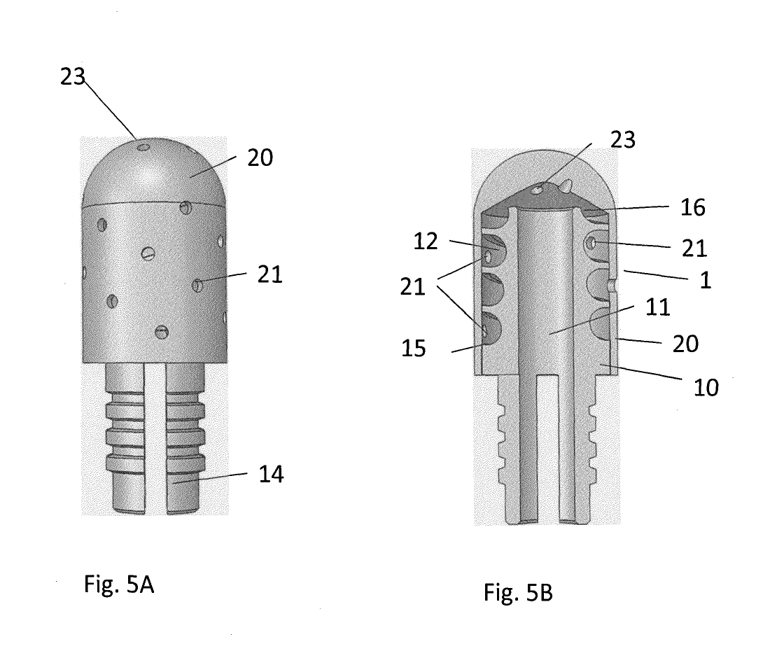

1. Irrigated ablation catheter comprising:
an elongated catheter shaft,
a deflectable section distal to the catheter shaft,
an ablation catheter tip distal to the deflectable section,
the ablation catheter tip including an irrigation fluid flow distributor 404 and a cap having a cylindrical side part and a tip, the irrigation fluid flow distributor having a distal end and a proximal end, whereby the irrigation fluid flow distributor has an inner channel and an outer channel, whereby the distal end of the inner channel is coupled to the distal end of the outer channel, and whereas the outer channel has a helical shape in the form of a double helix and is defined by the cylindrical part of the cap and the irrigation fluid flow distributor, whereby the cylindrical part of the cap is tightly slid over the irrigation fluid flow distributor and seals the outer channel of the flow distributor thus forming two helical cavities; whereby irrigation fluid exit ports are arranged on the cylindrical part of cap, said exit ports being arranged such that they follow a spiral course of the outer channel and whereby the cap has further irrigation fluid exit ports which are arranged on the tip of the cap.

2. Irrigated ablation catheter according to claim 1, wherein the cross section of the helical outer channel decreases from a distal end thereof to a proximal end thereof.

3. Irrigated ablation catheter according to claim 1, wherein both the cap and the irrigation fluid flow distributor are made of the same conductive material.

4. Irrigated ablation catheter according to claim 1, wherein the cap and the irrigation fluid flow distributor are made of different conductive material.

5. Irrigated ablation catheter according to claim 4, wherein the cap is made of Au or Pt/Ir and the irrigation fluid flow distributor is made of stainless steel.

6. Irrigated ablation catheter according to claim 14, wherein the cap is made of a conductive material and the irrigation fluid flow distributor is made of a non-conductive material.
